(19) United States
(12) Patent Application Publication
(10) Pub. No.: US 2009/0111783 A1
(43) Pub. Date: Apr. 30, 2009

(54) DERMATOLOGICAL COMPOSITIONS COMPRISING HYDROQUINONE, FLUOCINOLONE ACETONIDE AND TRETINOIN FOR TREATING THE HYPERPIGMENTATION OF PATHOLOGICAL SCARS

(75) Inventors: Isabelle PELISSON, Vallauris (FR); Andre Jomard, Saint Vallier De Thiey (FR)

Correspondence Address: BUCHANAN, INGERSOLL & ROONEY PC, POST OFFICE BOX 1404, ALEXANDRIA, VA, 22313-1404, US

(73) Assignee: GALDERMA S.A., CHAM (CH)

(21) Appl. No.: 12/257,667

(22) Filed: Oct. 24, 2008

Related U.S. Application Data
(63) Continuation of application No. PCT/FR2007/051193, filed on Apr. 27, 2007.

(30) Foreign Application Priority Data
Apr. 28, 2006 (FR) 0603879

(51) Int. Cl. A61K 31/573; A61P 17/02

(52) U.S. Cl. 514/171

(57) ABSTRACT

Dermatological medicament compositions contain a combination of hydroquinone, fluocinolone acetonide and tretinoin and are useful for the treatment of the hyperpigmentation of pathological scars, namely, hypertrophic scars and keloidal scars.

### CROSS-REFERENCE TO PRIORITY/PCT APPLICATIONS

This application claims priority under 35 U.S.C. §119 of FR 06/03879, filed Apr. 28, 2006, and is a continuation/national phase of PCT/FR 2007/051193, filed Apr. 27, 2007 and designating the United States (published in the French language on Nov. 8, 2007 as WO 2007/125262 A1; the title and abstract were also published in English), each hereby expressly incorporated by reference in its entirety and each assigned to the assignee hereof.

### BACKGROUND OF THE INVENTION

1. Technical Field of the Invention

The present invention relates to dermatological compositions comprising a combination of hydroquinone, fluocinolone acetonide and tretinoin for the treatment of the hyperpigmentation of pathological scars.

2. Description of Background and/or Related and/or Prior Art

The healing of a wound is a natural biological phenomenon which makes it possible, by repair and regeneration processes, to repair lesions.

The speed and the quality of the healing of a wound depend on the general condition of the organism affected, on the etiology of the wound, on the condition and location of the wound, and on the occurrence or non-occurrence of an infection, and also on the genetic factors causing or not causing a predisposition to disorders of healing.

Healing is the process which results in a scar. This process is also known as connective (or fibrous) organization of the inflammatory focus. The inflammatory reaction, by cellular and humoral mechanisms, induces the formation of an inflammatory granuloma which is gradually transformed into a regeneration blastema (or fleshy granulation) which constitutes the first stage of healing. The fleshy granulation is a transient newly formed connective tissue which will undergo significant modifications which ensure its transformation into a cicatricial fibrous tissue.

Inflammation is a dynamic process composed of a combination of vascular, cellular and humoral reactions triggered by any tissue lesion, whatever the cause (infectious, physical, chemical or ischemic). It makes possible the removal of the aggressive agent and cell debris and the repair of damaged tissue.

The healing process takes place in four main phases:

the initial vascular/exudative phase, which comprises active congestion of the vessels, an edema and the migration of the leukocytes towards of the site of the inflammation;

the phase of forming the inflammatory granuloma, which is converted into a regeneration blastema, also known as fleshy granulation;

the phase of cleaning (namely, the removal of the necrotic tissues, microorganisms, possible foreign bodies and the edema fluid), of inflammation and of epithelialization (namely, multiplication of the epidermal cells and end of healing);

the healing phase proper, which makes possible the change from a fleshy granulation to the cicatricial fibrous tissue (or scar).

Usually, a wound is healed after 10 days. Starting from the 60th day, the scar passes through a physiological hypertrophic phase, during which phase it will thicken and become connective and the neighboring tissues will become retractile. This hypertrophic phase is virtually complete after 1 year. Afterwards, the scar is no longer red or stiff and does not cause pain; it becomes flat.

However, in certain cases, healing does not take place as well and pathological scars are formed. The term “healing disorders” is then employed. These disorders are conventionally defined as disruptions of healing; they bring together two phenomena:

ulcers, which are an abnormality of healing where the wound becomes hollow and where the granulation tissue is not reconstructed. Hypotrophic or atrophic scars, resulting in particular from traumas but also from skin pathologies, such as acne vulgaris or chicken-pox, are hollow areas or ice-pick scars; their form is also due to an abnormality of healing (Topiramate and scars, Bharti Rakesh and Agarwal Lovedhi, Dermatology Online Journal, 11 (3), 42; Treatment of scars: a review, Alster et al., Ann. Plast. Surg., 1997, October, 39(4), 418-32);

hypertrophic scars and keloids (or “keloidal scars”), which are processes where the granulation tissue hyperproliferates in an abnormal fashion (Treatment of scars: a review, Alster et al., Ann. Plast. Surg., 1997, October, 39(4), 418-32).

Healing disorders thus bring together pathologies which are very different from the normal healing process.

The present invention is concerned with 2 types of pathological scars: “hypertrophic” scars and “keloid” or “keloidal” scars.

Whether hypertrophic or keloidal, these scars have as common origin an initial hyperplasic phase of high intensity and/or lengthy duration, which phase brings about an excess of dense fibrous tissue in the dermis. Pathological scars are large, swollen, red and hard, and itch.

The change in these scars over time makes it possible to distinguish a hypertrophic scar from a keloidal scar.

This is because:

hypertrophic scars spontaneously improve over time (in 2 or 3 years on average). They remain confined to the original site of the scar;

keloidal scars for their part do not have any tendency to spontaneously improve and remain stable, indeed even become worse, with time. Furthermore, this type of scar expands beyond the original site of the scar and affects the neighboring healthy tissues.

The cause or causes at the root of the formation of these pathological scars are still poorly known but there are a number of factors which favor their onset.

Exemplary thereof, among the risk factors for the formation of pathological scars, are:

race: individuals of the black or Asiatic race are much more subject to keloids than individuals of the white race;

age: frequent in children, hypertrophic scars are rare in elderly subjects;

location on the body: certain parts of the body are more prone to develop pathological scars, such as, for example, the sternum, neck, ear lobes or lower part of the face.

Intralesional excision or resection treatments for keloids in particular (in order not to again induce a lesion) exist in order to treat these pathological scars.

The treatment of hypertrophic and keloid scars is obviously not only surgical. As the cause of the hypertrophic scar is unknown, risks of recurrence after a simple surgical alteration to the scar exist. Surgery can certainly reduce the size of the scar when it is too large but it is then necessary to follow it, as rapidly as possible, with the following two methods, alone or in combination:

“pressure therapy”, carried out with made-to-measure compressive elastic clothing or also with silicone dressings with compression. It is highly effective, provided that it is applied permanently (day and night) for approximately 6 months, which is not always achievable;

“corticotherapy”, by injection inside the scar of prolonged-effect cortisone products. Due to the normal great hardness of these scars, the best method for injecting the product under pressure into the scar is to use a needleless device (“Dermo Jet”).

Treatments employing interferon also exist but, currently, whatever the treatment used, complete disappearance of the lesions is only rarely achieved.

Furthermore, in addition to the problems of size, form, pain and itching, hypertrophic scars present real problems to the patients who are affected by them. These scars are often unsightly due to their form but also because they are often hyperpigmented with respect to the surrounding tissues. These various symptoms can cause psychological distress to the patients affected by such scars and a true treatment is necessary.

### SUMMARY OF THE INVENTION

Surprisingly, it has now been determined that the combination of hydroquinone, tretinoin and fluocinolone acetonide makes it possible to efficiently treat the hyperpigmentation and to improve the flexibility of pathological scars and hyperpigmented scars.

The present invention thus features administration of a combination of hydroquinone, tretinoin and fluocinolone acetonide for the treatment of the hyperpigmentation of pathological scars, whether regime or regimen.

This is because such a combination makes it possible in particular to depigment pathological scars, for the purpose of obtaining a scar with a pigmentation almost homogeneous with the surrounding tissues.

According to the invention, the term “pathological scars” means hypertrophic scars and keloidal scars.

The term “hyperpigmented scars” means the scars formed with an excess of pigmentation.

Advantageously, the medicaments according to the present invention are suited for topical application.

The medicaments according to the present invention also comprise a physiologically acceptable medium, namely, a medium which is compatible with the skin, including the scalp, mucous membranes, hair, body hairs and/or eyes, and can constitute a dermatological composition.

The present invention also features formulation of a combination of hydroquinone, tretinoin and fluocinolone acetonide into cosmetic compositions useful for the depigmentation of pathological scars and in particular hypertrophic and keloidal scars.

### DETAILED DESCRIPTION OF BEST MODE AND SPECIFIC/PREFERRED EMBODIMENTS OF THE INVENTION

Hydroquinone is a known depigmenting agent. It is prepared by reduction of p-benzoquinone with sodium bisulfite. The chemical name of hydroquinone is 1,4-benzenediol.

Advantageously, the hydroquinone is present in the medicaments according to the present invention at a concentration of from 1% to 10% by weight, advantageously from 2% to 7% by weight, and more advantageously still approximately 4% by weight, with respect to the total weight of the medicament.

The chemical name of tretinoin is (all-E)-3,7-dimethyl-9-(2,6,6-trimethyl-1-cyclohexen-1-yl)-2,4,6,8-nonatetraenoic acid. This is an all-trans retinoic acid formed by the oxidation of the aldehyde group of retinene to give a carboxyl group. It is highly reactive towards light and moisture. It is a keratolytic agent.

In one specific embodiment, the tretinoin is present in the medicaments according to the present invention at a concentration of from 0.025% to 2% by weight, advantageously from 0.025% to 1% by weight, more advantageously still approximately 0.05% by weight, with respect to the total weight of the medicament.

The chemical name of fluocinolone acetonide is (6,11,16)-6,9-difluoro-11,21-dihydroxy-16,17-[(1-methylethylidene)bis(oxy)]pregna-1,4-diene-3,20-dione.

It is a white crystalline powder which is odorless and stable towards light. Fluocinolone acetonide is a fluorinated synthetic corticosteroid intended for a topical dermatological application and it is administered as an anti-inflammatory.

In another specific embodiment, the fluocinolone acetonide is present in the medicaments according to the present invention at a concentration of from 0.005% to 0.1% by weight, advantageously from 0.005% to 0.05% by weight, more advantageously still approximately 0.01% by weight, with respect to the total weight of the medicament.

Advantageously, the medicaments according to the present invention comprise sodium metabisulfite in order to prevent the hydroquinone from oxidizing.

Furthermore, the compositions as described above can comprise all the constituents normally present in the type of application envisaged.

The medicaments according to the present invention can comprise a large variety of additional components; in particular, they can be absorbents, abrasives, anti-acne agents, anti-foaming agents, antimicrobial agents, antioxidants, binders, biological additives, buffers, chelating agents, colorants, cosmetic astringents, cosmetic biocides, external analgesics, film-forming agents, fragrance components, opacifying agents, plasticizers, preservatives, other depigmenting agents, emollients, skin-protecting agents, solvents, solubilizing agents, surfactants, agents which absorb ultraviolet light, sunscreens, viscosity-increasing agents (aqueous or non-aqueous), humectants, sequestering agents, and the like.

These additional components can be present in the medicaments according to the present invention in an amount of from 0.001% to 20% by weight, with respect to the total weight of the medicament.

One skilled in this art will obviously take care to select the possible additional compounds and/or their amounts such that the advantageous properties of the medicaments according to the present invention are not completely or not substantially reduced by the envisaged addition.

The medicaments according to the present invention can be provided in any pharmaceutical dosage form. Normally the medicament will be provided in the cream form. The term “cream” means a water-based preparation for topical application. It corresponds to an emulsion, i.e., comprises at least one lipophilic phase and at least one hydrophilic phase.

The cream form can advantageously be prepared as indicated in WO 2004/037201, by a process comprising the stages of:

mixing the hydrophilic compounds with water in order to form an aqueous or hydrophilic phase;

mixing the hydrophobic compounds in order to form a hydrophobic phase;

mixing the hydrophobic and hydrophilic phases in order to form a two-phase mixture; and

adding an emulsifier to the two-phase mixture in order to form an emulsion.

Furthermore, they can comprise other normal ingredients of creams and be formulated in a manner well known to one skilled in the art.

Advantageously, the medicaments according to the present invention comprises at least one inactive ingredient selected from butylated hydroxytoluene, cetyl alcohol, citric acid, glycerol, glyceryl stearate, magnesium aluminum silicate, methyl gluteth-10, methylparaben, PEG-100 stearate, propylparaben, purified water, sodium metabisulfite, stearic acid and stearyl alcohol.

Advantageously, the medicaments according to the present invention correspond to the cream Tri-Luma® marketed by Galderma, as presented in Example 1.

In order to further illustrate the present invention and the advantages thereof, the following specific examples are given, it being understood that same are intended only as illustrative and in nowise limitative. In said examples to follow, all parts and percentages are given by weight, unless otherwise indicated.

### Example 1

### Composition of the Cream Tri-Luma®

The cream has the following formulation, as percentage by weight with respect to the total weight:

| | |
|---|---|
| magnesium aluminum silicate | 3.00% |
| butylated hydroxytoluene | 0.04% |
| cetyl alcohol | 4.00% |
| stearic acid | 3.00% |
| stearyl alcohol | 4.00% |
| methylparaben | 0.18% |
| propylparaben | 0.02% |
| Arlacel ® 165 [glyceryl | 3.50% |
| monostearate/PEG-100 stearate] | |
| methyl gluceth-10 | 5.00% |
| glycerol | 4.00% |
| tretinoin | 0.05% |
| fluocinolone acetonide | 0.01% |
| citric acid | 0.05% |
| hydroquinone | 4.00% |
| sodium metabisulfite | 0.20% |
| purified water | 68.95% |

Each patent, patent application, publication, text and literature article/report cited or indicated herein is hereby expressly incorporated by reference in its entirety.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A dermatological medicament composition comprising a combination of hydroquinone, tretinoin and fluocinolone acetonide, in amounts effective for the treatment of the hyperpigmentation of pathological scars selected from among hypertrophic scars and keloidal scars, formulated into a physiologically acceptable medium therefor.

2. The dermatological medicament composition as defined by claim 1, formulated for topical application.

3. The dermatological medicament composition as defined by claim 1, wherein the hydroquinone is present at a concentration of from 1% to 10% by weight, with respect to the total weight of the medicament composition.

4. The dermatological medicament composition as defined by claim 1, wherein the tretinoin is present at a concentration of from 0.025% to 2% by weight, with respect to the total weight of the medicament composition.

5. The dermatological medicament composition as defined by claim 1, wherein the fluocinolone acetonide is present at a concentration of from 0.005% to 0.1% 30 by weight, with respect to the total weight of the medicament composition.

6. The dermatological medicament composition as defined by claim 1, wherein the medicament composition is formulated as a cream.

7. The dermatological medicament composition as defined by claim 1, wherein the medicament composition comprises the following constituents, as percentages by weight with respect to the total weight thereof:

| | |
|---|---|
| magnesium aluminum silicate | 3.00% |
| butylated hydroxytoluene | 0.04% |
| cetyl alcohol | 4.00% |
| stearic acid | 3.00% |
| stearyl alcohol | 4.00% |
| methylparaben | 0.18% |
| propylparaben | 0.02% |
| Arlacel ® 165 [glyceryl | |
| monostearate/PEG-100 stearate] | 3.50% |
| methyl gluceth-10 | 5.00% |
| glycerol | 4.00% |
| tretinoin | 0.05% |
| fluocinolone acetonide | 0.01% |
| citric acid | 0.05% |
| hydroquinone | 4.00% |
| sodium metabisulfite | 0.20% |
| purified water | 68.95% |

8. The dermatological medicament composition as defined by claim 3, said hydroquinone concentration ranging from 2% to 7% by weight.

9. The dermatological medicament composition as defined by claim 8, said hydroquinone concentration being approximately 4% by weight.

10. The dermatological medicament composition as defined by claim 4, said tretinoin concentration ranging from 0.025% to 1% by weight.

11. The dermatological medicament composition as defined by claim 10, said tretinoin concentration being approximately 0.05% by weight.

12. The dermatological medicament composition as defined by claim 5, said fluocinolone acetonide concentration ranging from 0.005% to 0.05% by weight.

13. The dermatological medicament composition as defined by claim 12, said fluocinolone acetonide concentration being approximately 0.01% by weight.

14. A regime or regimen for the depigmentation of pathological scars selected from among hypertrophic scars and keloidal scars, comprising administering to a subject in need of such treatment, a thus effective amount of the dermatological medicament composition as defined by claim 1.
